(19) United States
(12) Patent Application Publication
(10) Pub. No.: US 2014/0242584 A1
(43) Pub. Date: Aug. 28, 2014

(54) GENOMIC DNA EXTRACTION REAGENT AND METHOD

(71) Applicant: SYNGENTA PARTICIPATIONS AG, Basel (CH)

(72) Inventors: Yanshan Ji, Durham, NC (US); Xiaoyin Fei, Durham, NC (US); Wenjin Yu, Durham, NC (US); Volker Mittendorf, Durham, NC (US)

(73) Assignee: SYNGENTA PARTICIPATIONS AG, Basel (CH)

(21) Appl. No.: 14/211,113

(22) Filed: Mar. 14, 2014

Related U.S. Application Data
(63) Continuation-in-part of application No. 14/171,816, filed on Feb. 4, 2014.
(60) Provisional application No. 61/769,925, filed on Feb. 27, 2013.

(51) Int. Cl. C12N 15/10; C12Q 1/68

(52) U.S. Cl. CPC C12N 15/1003; C12Q 1/6806
USPC 435/6.11; 435/6.12; 536/25.42; 252/192

(57) ABSTRACT

The present invention is directed to a genomic DNA extraction reagent and method for improved extraction of DNA from biological tissue. The extraction reagent of the invention is mixed with disrupted biological tissue to form a DNA extraction solute which is incubated in a DNA extraction step. The extraction reagent includes an alkali component to maintain the DNA extraction solute at a pH of about 10 to 14 substantially throughout the extraction step. The extraction solute is centrifuged to clarify the supernatant. The supernatant containing the extracted DNA is diluted with a neutralizing buffer resulting in a high throughout method of generating high quantities of high quality DNA. Major PCR inhibitors are managed with the unique chemical combinations of the DNA extraction reagent designed and optimized for extraction of DNA from plant tissue and cells.

### CROSS-REFERENCE TO RELATED APPLICATIONS

This application is a continuation in part of U.S. patent application Ser. No. 14/171,816 filed on Feb. 4, 2014, which claims priority to U.S. Provisional Application No. 61/769,925 filed on Feb. 27, 2013, which applications are hereby incorporated by reference.

### FIELD OF THE INVENTION

There is a need for low cost, high speed/throughput extraction of genomic DNA from animal or plant tissue or cells. The present invention encompasses a novel high pH DNA extraction reagent and a method of its use for obtaining high quality DNA from tissue or cells.

### BACKGROUND

There are a multitude of procedures involving obtaining, analyzing, and working with genomic DNA (gDNA). Automated high throughout laboratory procedures are critical for advancement of DNA focused technologies, e.g., in the pharmaceutical and agricultural industries. Traditional extraction of high quality gDNA from plant tissue is based on incubation of tissue material in various DNA extraction reagents that can include compounds that lyse cells and isolate DNA molecules from cellular debris. Following incubation, the DNA is pelleted and the pellet is re-suspended for use in, e.g. Polymerase Chain Reaction (PCR). Dellaporta et al. Plant Molecular Biology Reporter, Vol 1, No. 4 (1983). The pelleting step removes the compounds and cellular debris in the DNA extraction solute that are inhibitory to the PCR reaction. There are numerous methods in the art other than pelleting to remove the DNA from the extraction solute, including filtration and binding of DNA to solid matrix followed by washes. For example, exemplary methods for isolation of nucleic acids include those described in Sambrook & Russell, 2001; Ausubel et al., 1988; and Ausubel et al., 1999. A primary method employed for extracting genomic DNA (gDNA) from plants employs cetyltrimethylammonium bromide (CTAB) to precipitate nucleic acids and acidic polysaccharides from solutions of low ionic strength, but can also be used to remove polysaccharides and proteins from solutions of higher ionic strength (e.g., <0.7 M NaCl; see Sambrook & Russell, 2001; see also Murray & Thompson, 1980).

Similarly as discussed with Dellaporta, drawbacks of the CTAB method are that in order to recover the nucleic acids from high ionic strength solutions, a subsequent treatment with organic solvents and alcohol precipitation and/or purification over a cesium chloride gradient is typically required. Such steps are not desirable for a high-throughput method, not only because of the labor, time, and expense involved, but also because if these steps are not taken, the nucleic acid preparation is likely to be contaminated with various inhibitors of downstream analyses.

An alternative method for isolating plant gDNA is disclosed in Kotchoni & Gachomo (2009) Mol Biol Rep 36:1633-1636. This multi-step method involves grinding plant tissue, incubating the same in a mixture of sodium dodecyl sulfate (SDS) and sodium chloride, spinning down insoluble aggregates, transferring the nucleic acid-containing supernatant to a new vessel, isopropanol precipitation of nucleic acids, re-spinning down the precipitated nucleic acids, performing an ethanol wash, spinning down the washed nucleic acids yet again, drying the nucleic acids, and dissolving the same in a buffer of choice. Given the numerous steps and manipulations required, this method is also largely unsuitable for automation. Additionally, many inhibitors of DNA polymerases are typically present in the re-suspended DNA, such that the DNA quality is generally not well suitable for quantitative PCR.

A third method for isolating plant gDNA is disclosed in Dilworth & Frey (2000) Plant Molecular Biology Reporter 18:61-64. This method employs Proteinase K and a detergent (e.g., polysorbate 20), but requires several incubations at elevated temperatures (e.g., 65° C.). It avoids the alcohol precipitation steps of some of the other methods, but generally the DNA yield is low and quality is poor, leading to unreliable PCR performance even using “regular” (i.e., non-quantitative) PCR. Additionally, since proteinase K is the only reagent that can remove potential inhibitors, no protein inhibitors are typically present in the isolated DNA. The cost of the enzyme itself can negatively impact the usefulness of this method in a high-throughput process. Of course, high per-run costs are also associated with methods that are based on using solid supports such as silica-based supports or magnetic beads to isolate gDNA.

As stated, the disadvantage of these traditional methods of obtaining high quality DNA from plant tissue is that they are time consuming and expensive, and therefore not suitable for high throughput processes.

A number of publications describe materials and methods for high throughput extraction of DNA from tissue in an attempt to increase efficiency and reduce cost. Wang et al. Nucleic Acids Research, 21, 4153-4154 (1993), Porcar et al. J. Sci. Food, Agric 87:2728-2731 (2007) and Montero-Pau, incubate tissue in a NaOH extraction reagent followed by a salt solution. Van Post et al Euphytica 130:255-260, 2003, Paris, Carter Plant Mol. Biol Reporter 18:357-360 (2000), and Montero-Pau Limnol. Oceanogr, Methods 6:218-222 (2008) extract DNA from tissue in a NaOH followed by incubation in Tris-HCl buffer and EDTA. Although these methods reduce the number of steps required to extract genomic DNA and the extracted DNA may be suitable for qualitative (presence or absence of DNA) PCR, it is unsuitable for quantitative PCR (qPCR) and applications that require larger amounts of high quality DNA. Osmundson et al. Molecular Ecology Resources 13:66-74 (2013). The publication WO2014/018195 (the '195 publication) describes a lysis buffer providing a low salt concentration chemical environment that is suitable for the activity and stability of DNA polymerase in PCR, wherein pH is required to be less than 10. The problem with this lysis buffer is that the resulting supernatant containing the DNA is diluted a minimum of 100 times in order to reduce the contaminant effect on PCR. Contamination can include the preparation reagents themselves as well as components of the plant tissues and/or cells that remain in the isolated gDNA sample. In particular, isolation of plant gDNA frequently results in the presence of high levels of polysaccharides, polyphenols, pigments, and/or other secondary metabolites (see Wen & Deng, 2002), the presence of which can make gDNA preparations unusable in downstream analyses (see Michiels et al., 2003; Qiang et al., 2004).

There remains a need for a quick, efficient, and cost effective gDNA extraction reagent and method for obtaining large quantities of high quality gDNA across different organisms.

There further remains a need for a quick, efficient, and cost effective DNA extraction reagent and method for obtaining large quantities of high quality genomic DNA across different plant species and from different plant parts.

### SUMMARY

High throughput and cost effective gDNA extraction is critical for many pharmaceutical and biotechnology applications. A number of critical genetic tools require high quantities of high quality gDNA. For example, real-time PCR for quantitative DNA (qPCR) analysis requires sufficient amounts of extracted gDNA and is also sensitive to DNA quality. qPCR requires at least 90% amplification efficiency to ensure that the limit of detection (LOD) for qPCR is reached (typically about 0.1%).

The present invention includes a novel gDNA extraction reagent and method that provides a DNA high salt extraction solute also including SDS that is maintained at a high pH during the extraction step. Preferably, the extraction solute is maintained at a pH of at least 12 and a salt concentration of at least 0.3 mM during the extraction step. The DNA extraction reagent of the invention is designed to lyse plant cells, denature cellular proteins, and release gDNA. While not wishing to be bound by any particular theory of operation, the extraction reagent is also believed to bind to lipids and denatured proteins, especially nucleoprotein in the plant chromosomes.

The present invention includes a novel gDNA extraction reagent and a fast and high throughput method to extract high quality DNA from animal or plant tissue or cells. The DNA extraction reagent of the invention is multifunctional. When mixed with plant or animal tissue or cells to form a DNA extraction solute, it causes cell lysis, disrupts and denatures DNA-protein and other macro-molecule complexes, frees DNA from other macromolecules, and precipitates otherwise deactivates major PCR inhibitors coming from biological materials.

According to the present invention, the gDNA extraction step generates genomic DNA present in the supernatant that is sufficiently low of contaminants and of sufficient quantity for use in qPCR.

The invention includes a gDNA extraction reagent comprising an alkali, a detergent, salt, and optionally a water soluble polymer such as polyvinylpyrrolidone (PVP).

The invention includes a gDNA extraction reagent comprising an alkali, a surfactant, salt, and optionally a water soluble polymer such as polyvinylpyrrolidone (PVP), wherein the extraction reagent is mixed with plant or animal tissue or cells to form a DNA extraction solute comprising cellular and tissue materials.

The invention includes a gDNA extraction reagent comprising a high pH strong base, a surfactant, and a salt.

The invention includes a gDNA extraction reagent comprising a strong base pH of at least 10, a surfactant, and a salt.

The invention includes a DNA extraction reagent as described above, wherein the water soluble polymer such as polyvinylpyrrolidone (PVP) is optional.

The invention also includes a dilution buffer that is intended to lower the pH of the gDNA solution, also referred to as the DNA extraction solute, to in some embodiments less than 10, in some embodiments in less than about 9.5, in some embodiments less than 9.0, in some embodiments less than 8.5, in some embodiments less than 8.0, and in some embodiments less than 7.5. In some embodiments, the pH of the DNA extraction solute upon adding the dilution buffer is about 7.0, 7.5, or 8.0.

The method of the invention includes obtaining pulverized, macerated, or otherwise disrupted animal or plant tissue or cells and mixing the DNA extraction reagent of the invention with the disrupted tissue or cells to form a DNA extraction solute and incubating the solute for a period of time. The gDNA in the supernatant is used directly in qPCR or other DNA amplification processes or analyses that require high quantities of high quality DNA.

The method of the invention includes obtaining pulverized, macerated, or otherwise disrupted animal or plant tissue and mixing DNA extraction reagent of the invention with the disrupted tissue to form a DNA extraction solute and incubating the solute for a period of time in a DNA extraction step, spinning the solute and using the supernatant containing the DNA in PCR or other DNA amplification processes or analysis that require high quantities of high quality gDNA.

The method of the invention includes obtaining pulverized, macerated, or otherwise disrupted animal or plant tissue and mixing the DNA extraction reagent of the invention with the disrupted tissue or cells to form a DNA extraction solute and incubating the solute for a period of time in a DNA extraction step, spinning by centrifugation the solute, collecting the clarified supernatant and then diluting the clarified supernatant in a neutralizing buffer and using the neutralized supernatant in PCR or other DNA amplification processes or analysis that require high quantities of high quality gDNA.

The method of the invention includes obtaining pulverized, macerated or otherwise disrupted animal or plant tissue or cells and mixing the DNA extraction reagent of the invention with the disrupted tissue or cells to form a DNA extraction solute, which is maintained at a pH of at least 10, preferably of at least 11, more preferably of at least 12, even more preferably of at least 13, and most preferred a pH of 14. After incubating the DNA extraction solute for a period of time in the DNA extraction step, gDNA is released from the tissue and cells and further from macro-molecule complexes and is subsequently collected along with the supernatant clarified by centrifugation. This high quality gDNA is used directly in PCR, or diluted preferably no more than 5 fold or more preferably diluted no more than 10 fold before using in PCR or other DNA amplification method.

The method of the invention includes obtaining pulverized, macerated or otherwise disrupted animal or plant tissue or cells and mixing the DNA extraction reagent of the invention with the disrupted tissue or cells to form a DNA extraction solute, wherein extraction reagent includes NaOH in a range of 0.1M to 0.2M and wherein during the DNA extraction step the pH of the solute is at least 10, preferably at least 11, more preferred at least 12, even more preferred at least 13, and most preferred a pH of 14. It is understood that collection of the supernatant may encompass using the same container in which the supernatant is clarified or transferring the clarified supernatant to another container.

It is understood that the high quality gDNA obtained using the DNA extraction reagent and method of the invention can be used for processes and activities that do not require the high quality DNA, e.g. traditional +/−PCR.

The method of the invention includes incubating animal or plant tissue or cells in the DNA extraction reagent of the invention, wherein the DNA extraction reagent of the invention comprises 0.1M NaOH, 0.1% SDS, 0.3M NH₄Ac, and optionally 1% PVP-40 and is mixed with disrupted animal or plant tissue or cells to form a tissue DNA extraction solute and incubated for a time in the DNA extraction step. The clarified supernatant is then diluted in a neutralizing buffer about 50 fold or less, preferably about 45 or less, more preferably about 40 fold or less, even more preferably 35 fold or less, even more preferably 30 fold or less, even more preferably 20 fold or less, even more preferably 15 fold or less, and most preferably 10 fold or less.

The method of the invention includes incubating disrupted animal or plant tissue or cells in the DNA extraction reagent of the invention to form a tissue DNA extraction solute and incubated for a time in what is referred to as the DNA extraction step. DNA is obtained from the supernatant of the DNA extraction solute and diluted in a neutralizing buffer up to about 1000 fold or more.

The method of the invention includes extracting gDNA from plant or animal tissue or cells in the DNA extraction step of the invention, and using the DNA in qPCR, isothermal DNA amplification or in other DNA amplification processes or analysis that requires high quantities of high quality DNA.

The method of the invention wherein the dilution buffer neutralizes the remaining strong base in the supernatant to a pH of 10 or less. The method of the invention includes adding the dilution buffer of the invention to the DNA extraction solute to lower the pH of the gDNA solution or DNA extraction solute to in some embodiments less than 10, in some embodiments less than about 9.5, in some embodiments less than 9.0, in some embodiments less than 8.5, in some embodiments less than 8.0, and in some embodiments less than 7.5. In some embodiments, the pH of the DNA extraction solute upon adding the dilution buffer of the invention is about 7.0, 7.5, or 8.0. A preferred embodiment of the invention includes adding a dilution buffer of the invention to the DNA extraction solute to lower the pH of the gDNA solution or DNA extraction solute to a range of about 8 to 9.

The DNA extraction reagent and method of the invention include a strong base in combination with SDS to more efficiently lyse cell, disintegrate DNA-protein complexes by denaturing and/or binding. Addition of salt precipitates the SDS-protein polysaccharides out of solution.

The DNA extraction reagent and method of the invention include a strong base that is NaOH in combination with SDS and NH₄Ac to more efficiently lyse cell, disintegrate DNA-protein complexes by denaturing and/or binding, wherein the strong base maintains the DNA extraction solute preferably at least a pH of 10 during the DNA extraction step, more preferably at least 11, even more preferably at least 12, yet more preferably at least 13, and most preferably a pH of 14.

The method of the invention includes extracting DNA from plant or animal tissue or cells in a DNA extraction step, wherein the DNA extraction reagent of the invention is mixed with disrupted animal or plant tissue or cells to form a DNA extraction solute. The DNA extraction solute is incubated for a period of time in the DNA extraction step and DNA is obtained from the supernatant thereof and diluted about 10 fold or less in a dilution buffer, wherein the dilution buffer neutralizes the remaining strong base in the supernatant and using the diluted supernatant in PCR, in qPCR, isothermal DNA amplification, or in other DNA amplification processes or analysis that requires high quantities of high quality gDNA.

In some embodiments, the presently disclosed subject matter also provides methods for isolating gDNA from biological material using the compositions disclosed herein. Thus, in some embodiments the presently disclosed methods comprise (a) contacting a sample comprising gDNA with a first solution comprising hydroxide and a detergent or surfactant under conditions and for a time sufficient to degrade a cell wall, a cell membrane, a nuclear membrane, or combinations thereof, and/or to denature the gDNA; (b) mixing into the solution resulting from step (a) a second dilution buffer solution with sufficient buffering capacity to reduce the pH of the solution to less than 10, thereby producing a neutralized preparation; (c) centrifuging the sample at a speed and for a length of time sufficient to clarify the neutralized preparation; and (d) removing insoluble material from the neutralized and clarified preparation, whereby a solution of gDNA is produced.

In some embodiments, the presently disclosed subject matter also provides methods for isolating gDNA from biological material using the compositions disclosed herein. Thus, in some embodiments the presently disclosed methods comprise (a) contacting a sample comprising gDNA with a first solution comprising hydroxide, a detergent or surfactant, and a salt under conditions and for a time sufficient to degrade a cell wall, a cell membrane, a nuclear membrane, or combinations thereof, and/or to denature the gDNA; (b) mixing into the solution resulting from step (a) a second dilution buffer solution with sufficient buffering capacity to reduce the pH of the solution to less than 10, thereby producing a neutralized preparation; (c) centrifuging the sample at a speed and for a length of time sufficient to clarify the neutralized preparation; and (d) removing insoluble material from the neutralized and clarified preparation, whereby a solution of gDNA is produced.

The invention further includes a kit containing the DNA extraction reagent of the invention and may further comprise a dilution buffer.

### BRIEF DESCRIPTION OF THE FIGURES

FIG. 1 displays an analysis of DNA extracts showing allelic calls for DNA extracted from corn tissue. FIG. 1 illustrates a typical clustering data set, showing clear clustering and separation between clusters. A negative control “x” is shown outside these clusters and represents no DNA template for PCR reaction. If a data point does not amplify and/or fit within a cluster, these data points are referred to as “missing” or “unscoreable” data. A high percentage of missing data indicates inferior DNA quality and/or quantity not suitable for PCR or qPCR, with normal assay and PCR analysis.

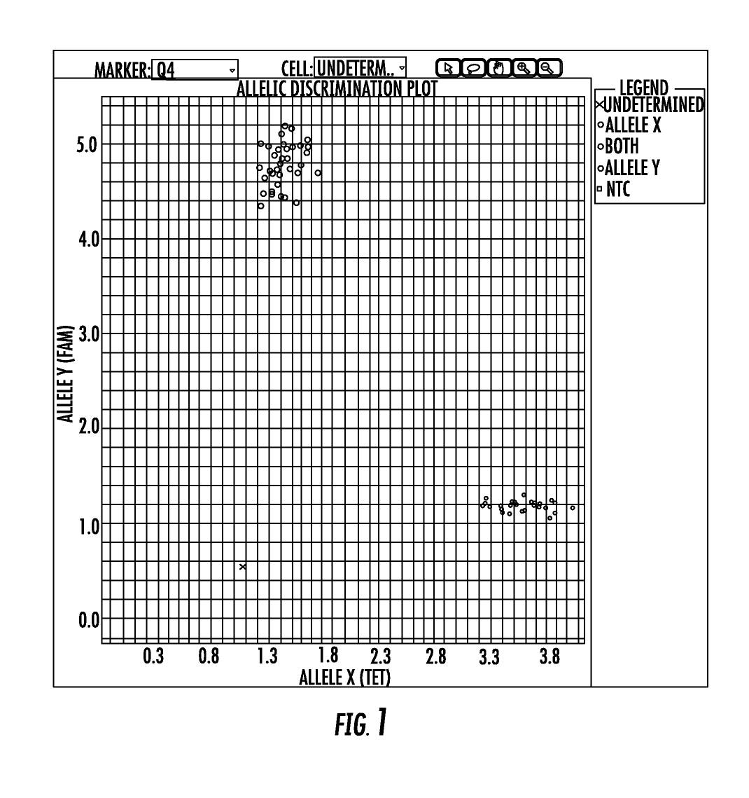

FIG. 2 displays an analysis of DNA extracts showing allelic calls for DNA extracted from soy tissue.

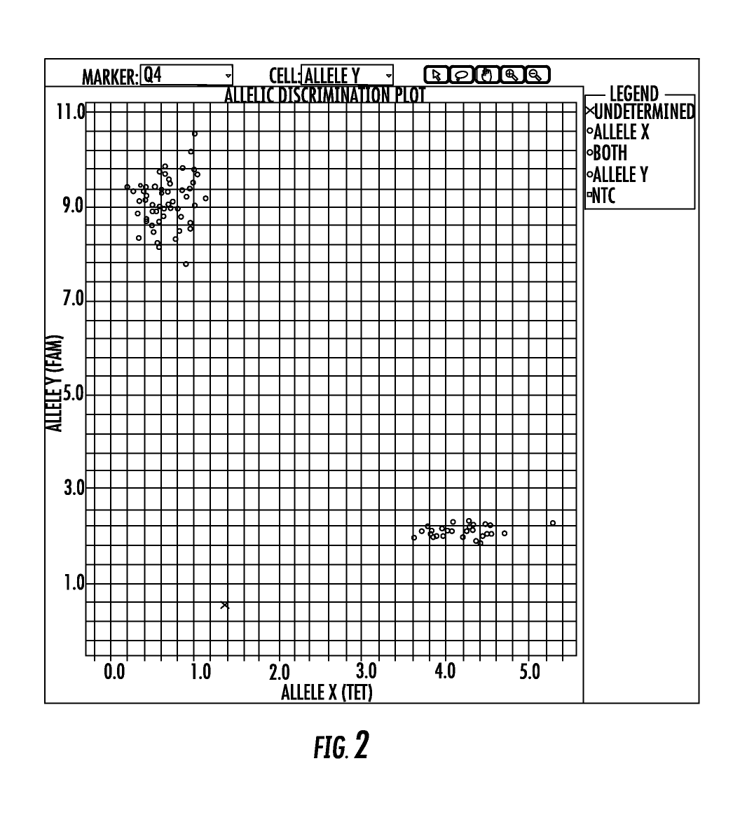

FIG. 3 displays an amplification plot showing good quality DNA generated by using the DNA extraction reagent and method of the invention.

FIG. 4 shows the impact of alkali and dilution on Ct value.

FIG. 5 shows the impact of NH₄Ac and dilution on Ct value.

FIG. 6 shows the impact of shows the impact various dilutions of the supernatant of the DNA extraction solute on the Ct value using the preferred 40 mM Tris-HCl at pH 7.5. DNA in supernatant at dilution 1:5 or less is acceptable for qualitative analysis, such as +/− analysis.

FIG. 7 shows the impact of on Ct value of different Tris-HCl dilution buffer concentrations for a 1:10 dilution of the supernatant. 40 mML Tris-HCl at pH7.5 was selected as the preferred condition. However, all depicted concentrations of Tris-HCl dilution buffer worked well at each pH.

### DETAILED DESCRIPTION OF THE INVENTION

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the articles “a”, “an”, and “the” refer to “one or more” when used in this application, including in the claims. For example, the phrase “a symptom” refers to one or more symptoms. Similarly, the phrase “at least,” for example “at least 1,” when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the phrase “biological material” refers to biological materials in any matrix, including but not limited to tissue isolated from a living multi-cellular organism, a culture of single-celled organisms, a soil or water sample, a food or feed sample, animal, human, or plant tissue culture, clinic samples, seeds, and/or seed powder.

Many cell contaminants need to be removed in order to produce a high quality DNA preparation, including secondary metabolites, enzymes, as well as polyphenols and proteases left after standard treatment with detergents, salts, and PVP. In addition, the reagents themselves are a source of DNA contamination. Thus, it is currently understood in the art that in order to prepare high quality DNA it is necessary to precipitate the DNA into a pellet or remove the DNA from solution containing the contaminants by passing over a membrane or other absorbing structure.

The invention therefore includes a DNA extraction reagent comprising a high pH strong base, a surfactant, salt, and optionally a water soluble polymer such as polyvinylpyrrolidone (PVP).

In accordance with the present invention, a DNA extraction reagent is used in a rapid, gDNA extraction process, wherein high quality DNA is obtained.

The phrase “high throughput” (HT) refers to extracting large quantities of high quality genomic DNA from animal or plant tissue or cells by mixing a multi-functional extraction reagent of the invention with tissue a sample, referred to herein after as the plant extraction solute, thereby causing extraction of genomic DNA in a rapid, extraction process.

The phrase “high quality” refers to meeting the criteria and requirements for quantitative PCR analysis, which may include: 1) Most genomic DNA fragments in the extract should have a molecular weight higher than the molecular weight of the amplicons of the PCR, and 2) substantial absence of co-extracted compounds in a DNA extracts that would impair the efficiency of the PCR amplification.

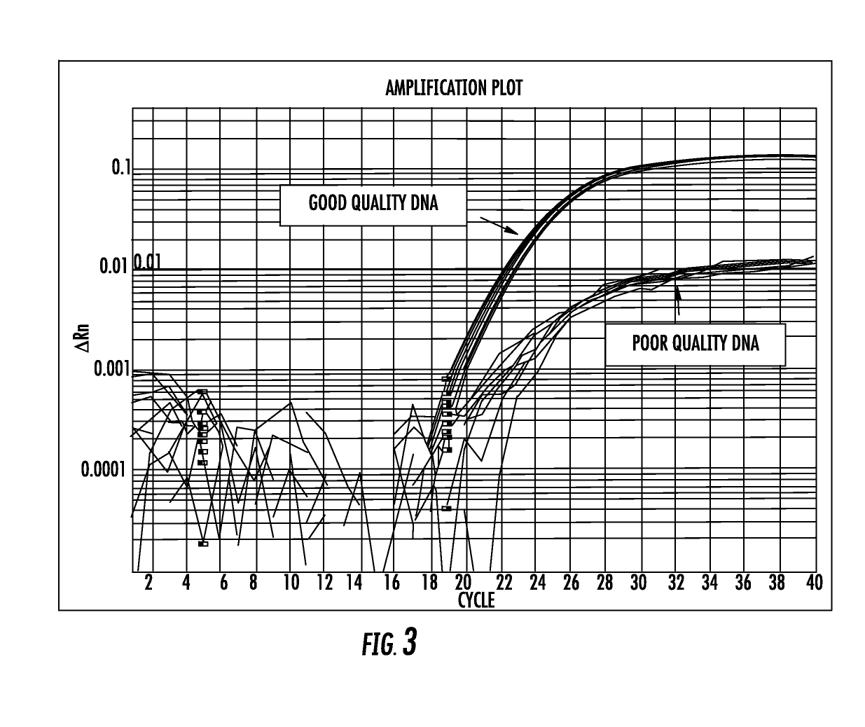

Referring to FIG. 3, high quality refers to a robust amplification as shown, wherein multiple replicates are reliably amplified with small variation of Ct values between the replicate, wherein Ct value is low indicating relative high concentration of DNA.

Further dilutions of DNA sample resulting in low concentration of DNA results in high Ct value in qPCR analysis and high “missing data” in genotyping analysis.

The term “neutralization” or “neutralizing” refers to a chemical reaction in which an acid and a base react to form a salt. Water is frequently, but not necessarily, produced as well. Neutralizations with Arrhenius acids and bases always produce water where acid-alkali reactions produce water and a metal salt. Neutralization reactions do not necessarily imply a resultant pH of 7. The resultant pH will vary based on the respective strengths of the acid and base reactants.

The term “dilution” refers to adding additional solvent to a solute, such as water, in order to make the solute less concentrated. The DNA extraction reagent and method of the present invention is characterized, in part, by the solute or supernatant being diluted a small amount compared to comparable methods currently known in the art, due to the supernatant containing high quantities of high quality gDNA and low quantities of inhibiting compounds typically generated during the extraction process.

As used herein, the phrase “Ct value” refers to “threshold cycle”, which is defined as the “fractional cycle number at which the amount of amplified target reaches a fixed threshold”. In some embodiments, it represents an intersection between an amplification curve and a threshold line. The amplification curve is typically in an “S” shape indicating the change of relative fluorescence of each reaction (Y-axis) at a given cycle (X-axis), which in some embodiments is recorded during PCR by a real-time PCR instrument. The threshold line is in some embodiments the level of detection at which a reaction reaches a fluorescence intensity above background. See Livak & Schmittgen (2001) 25 Methods 402-408. It is a relative measure of the concentration of the target in the PCR. Generally, good Ct values for quantitative assays such as qPCR are in some embodiments in the range of 10 to 40 for a given reference gene, preferably in the range of 10 to 30, more preferably in the range of 10 to 28, even more preferably in the range of 10-26, more preferably in range of 10-24, and most preferably in range of 10-20. Ct levels are inversely proportional to the amount of target nucleic acid in the sample (i.e., the lower the Ct level the greater the amount of detectable target nucleic acid in the sample).

Additionally, good Ct values for quantitative assays such as qPCR show a linear response range with proportional dilutions of target gDNA and such values represent high quality target nucleic acid (DNA), which in the case of the invention was extracted from animal or plant tissue or cells.

In some embodiments, qPCR is performed under conditions wherein the Ct value can be collected in real-time for quantitative analysis. For example, in a typical quantitative PCR experiment, DNA amplification is monitored at each cycle of PCR during the extension stage. The amount of fluorescence generally increases above the background when DNA is in the log linear phase of amplification. In some embodiments, the Ct value is collected at this time point.

The term “limit of detection” (LOD) refers to the lowest concentration of analyte in a sample, which can be reliably detected, but not necessarily quantified. The lower the LOD the higher the sensitivity.

The term “sensitivity” is used as a general term, and is often used interchangeably with LOD. The sensitivity or detection sensitivity will be lower if the DNA has to be diluted more in order to remove or reduce the negative effects of impurities in the DNA sample. For example, assume there are 10,000 copies of a target gene in 1 μl of DNA supernatant, a TaqMan PCR assay can detect as low as 1 molecule. If DNA sample needs to be diluted 1:10 dilution to obtain suitable PCR starting sample then the best sensitivity of the PCR using 1 μl diluted DNA is 0.1% (10000 copies/10=1000 copies, 1000×0.1%=1 copy or molecule). If a DNA needs 1:50 dilution then the best sensitivity will be compromised to 0.5% meaning the PCR analysis is less sensitive. A sensitivity of 0.1% is acceptable for many applications of qPCR.

The term “amplification efficiency” refers to the rate of amplification that leads to a theoretical slope of −3.32 with an efficiency of 100% in each cycle. Amplification efficiency can be calculated by the formula: Efficiency =10^{(-1/slope})−1.

The amplification efficiency from qPCR testing using series dilution shows that a slope of ≧−3.6 can translate to an efficiency ≧0.9 or ≧90% using the above equation. If the PCR assay is “normal”, than either efficiency <90% or >110% (slope >−3.1) means the DNA quality is not suitable for qPCR analysis, although the DNA may still be useful for +/− analysis using such techniques as gel-based PCR analysis.

The term “buffer” means an aqueous solution consisting of a mixture of a weak acid and its conjugate base. The pH of a solution changes very little when a small amount of strong acid or base is added to it. Buffer solutions are used as a means of keeping pH at a nearly constant value in a wide variety of chemical applications.

The DNA extraction reagent of the invention is multi-functional serving to lyse cells, disrupt and denature DNA-protein and other macro-molecule complexes, removal of major PCR inhibitors, to obtain the sufficient quantities of high quality DNA in the supernatant for a rapid, extraction process.

The DNA extraction reagent of the invention comprising a high pH strong base, a surfactant, salt, and a water soluble polymer such as polyvinylpyrrolidone (PVP).

The DNA extraction reagent of the invention includes an alkali, such as NaOH, a detergent such as sodium dodecyl sulphate (SDS), a salt such as NH₄Ac, and optionally PVP-40.

In accordance with the present invention, a DNA extraction reagent is used in a process, wherein cells are incubated in a DNA extraction reagent in a DNA extraction step and high quality gDNA is obtained from supernatant thereof.

In accordance with the present invention, a DNA extraction reagent is used in a process, wherein cells are incubated in a DNA extraction reagent in a DNA extraction step and high quality DNA is obtained from supernatant thereof, wherein the DNA extraction reagent is kept at a high pH of at least 10, preferably of at least 11, more preferably of at least 12, even more preferably of at least 13, or most preferably a pH of 14 during the DNA extraction step.

In accordance with the present invention, a DNA extraction reagent is used in a process for extraction of gDNA from plant or animal tissue.

The extraction reagent of the invention may be used to extract gDNA from different plant species and from different tissues of a plant species.

The extraction reagent of the invention may be used to extract gDNA from the tissue and/or cells of many different plants, including but not limited to sorghum, wheat, sunflower, tomato, potato, cotton, rice, soybean, sugar beet, sugarcane, tobacco, barley, oilseed rape, algae, maize, and including transgenic plants.

In accordance with the present invention, a DNA extraction reagent is used in a process, wherein disrupted tissue or cells are incubated in the extraction reagent of the invention and high quality DNA is obtained from the supernatant thereof and wherein the supernatant is diluted in a neutralizing buffer and used in a DNA amplification reaction such as PCR and/or qPCR.

DNA extraction method of the invention includes cell lysis and removal of contaminants resulting in DNA that is sufficiently low of contaminants allowing for effective qPCR amplification and analysis.

Disrupting plant or animal tissue may be accomplished by any number of means known in the art including viral, enzymatic or osmotic mechanisms augmented by physical methods such as blending, grinding or sonicating.

In one embodiment of the DNA extraction method of the invention tissue and cells are disrupted by adding beads to a container containing such tissue and/or cells. It is preferred that the tissue and cell disruption step does not include the use of liquid media, but that it is done “dry” or “frozen” (for fresh tissue). The container is shaken for a period of time and then spun. Once the tissues and cells are disrupted, DNA extraction reagent is added to the container to begin the DNA extraction step.

The genomic DNA extraction reagent of the invention includes a strong base component or alkali. Alkalis that may be used according to the invention include potassium hydroxide, (KOH), metal hydroxide, alkali salts, and sodium hydroxide (NaOH), or combinations thereof.

It is understood that the alkaline component of the DNA extraction reagent may be selected from a number of alkali compounds, which all would be within the scope of the invention, provided the alkali produces a DNA extraction reagent with a high basic pH as described above.

The alkaline component of the DNA extraction reagent of the invention provides a DNA extraction solute having a pH in the range of 10 to 14 and that is maintained throughout the DNA extraction step.

The alkaline component of the DNA extraction reagent of the invention provides a DNA extraction solute having a pH preferably in the range of 11 to 14 that is maintained throughout the DNA extraction step.

The alkaline component of the DNA extraction reagent of the invention provides a DNA extraction solute having a pH even more preferably in the range of 12 to 14 that is maintained throughout the gDNA extraction step.

The alkaline component of the DNA extraction reagent of the invention provides a DNA extraction solute having a pH yet more preferably in the range of 13 to 14 that is maintained throughout the gDNA extraction step.

The alkaline component of the DNA extraction reagent of the invention provides a DNA extraction solute having a pH yet more preferably of 14 that is maintained throughout the gDNA extraction step

According to the invention, the alkali may be used in the DNA extraction reagent in concentrations of about 0.1M to 0.2M.

The DNA extraction reagent does not contain components that would neutralize the alkali or meaningfully lower the pH during the gDNA extraction step.

Membrane lipids and oils are removed by adding a detergent or surfactant in addition to alkali. The term “surfactant” refers to a surface active agent that generally comprises hydrophobic and hydrophilic portions. Examples of surfactants include, but are not limited to, detergents and bile salts. There are numerous surfactants that may be incorporated into the DNA extraction reagent of the invention to address lipid and oil contamination, including ionic detergents such as anionic and cationic detergents as well as non-ionic and zwitterionic detergents. Sodium dodecyl sulphate (SDS) can be used as an anionic detergent in the extraction reagent of the invention, wherein ethyl trimethyl ammonium bromide can be used as a cationic detergent. Other detergents that may be used in accordance with the invention are Triton X-100, Triton X-114, NP-40, Tween 20, Tween 80, Octyl glucoside, Octyl thioglucoside, 3-[(3-cholamidopropyl)dimethylammonio]-1-propanesulfonate CHAPS, cetyltrimethylammonium bromide (CTAB) and combinations thereof. Some detergents such as SDS can also bind proteins.

Alkali and salts are traditionally used at relatively high concentrations in order to disrupt protein and DNA molecules, including but not limited to changing secondary, tertiary, and quaternary structures. Salts neutralize the charge of a DNA's sugar phosphate backbone, making them less hydrophilic (less soluble in water). Salts are also used in extraction methods to remove polysaccharides and proteins as co-contaminants. Exemplary salts include, but are not limited to ammonium acetate (NH₄Ac), sodium chloride (NaCl), potassium phosphate, sodium bicarbonate, sodium acetate (NaAc), and potassium acetate (KAc), and combinations thereof.

Polyvinylpyrrolidone (PVP) can be used according to the invention to manage phenolic compounds which may be co-contaminants and major inhibitors to PCR.

In some applications, cell or tissue extracts may be low in phenolic compounds such that PVP may not be needed as a component of the DNA extraction reagent of the invention. Thus, PVP is an optional component of the DNA extraction reagent of the invention.

Experiments showed that the concentrations of DNA extraction reagent components may vary with ranges and still maintain ability to generate a clarified supernatant containing high quantities of high quality gDNA as described herein. Several preferred embodiments of the DNA extraction reagent of the invention are listed below.

An embodiment of the DNA extraction reagent of the invention comprises at least 0.1M NaOH, 0.1%, SDS, 0.3M NH₄Ac, and 1% PVP-40.

Another embodiment of the DNA extraction reagent of the invention comprises NaOH at 0.1M to 0.2M, 0.1%, SDS, and 0.3M NH₄Ac.

Yet another embodiment of the DNA extraction reagent of the invention comprises at least 0.1M NaOH, 0.1%, SDS, and at least 0.3M NH₄Ac.

Another embodiment of the DNA extraction reagent of the invention comprises 0.1M NaOH, 0.1%, SDS, and 0.3M to 0.6M NH₄Ac.

Another embodiment of the DNA extraction reagent of the invention comprises NaOH at 0.1M to 0.2M, 0.1%, SDS, and 0.3M to 0.6M NH₄Ac.

Another embodiment of the DNA extraction reagent of the invention comprises 0.1M NaOH, 0.1%, SDS, and 0.1M to 0.6M NH₄Ac

Still another embodiment of the DNA extraction reagent of the invention comprises 0.1M to 0.2M NaOH, 0.1%, SDS, and 0.1M to 0.6M NH₄Ac.

Another embodiment of the DNA extraction reagent of the invention comprises at least 0.1M NaOH, 0.01% to 1.0%, SDS, and 0.3M to 0.6M NH₄Ac.

Another embodiment of the DNA extraction reagent of the invention comprises 0.1M to 0.2M NaOH, 0.01% to 1.0%, SDS, 0.3M to 0.6M NH₄Ac.

The DNA extraction reagent of the invention optionally includes about 0.1%-5% PVP-40 in the DNA extraction reagent.

A preferred DNA extraction reagent of the invention comprises at least 0.1M NaOH, and at least 0.3M NH₄Ac and further comprising an effective amount of SDS.

The general method of the invention includes securely seal sample block containing 1 steel bead and 4 leaf discs/well frozen or lyophilized. Each disc is approximately 6 mm in diameter.

Shake the block for 2 minutes in a paint shaker or equivalent grinder, such as a Kleco grinder.

Centrifuge the block for 1 minute at about 3200 RCF at RT.

Dispense 200 μl of DNA extraction reagent into each well to form the DNA extraction solute. Re-seal the block and shake for about 1 minute in the grinder.

Incubate the solute at RT for 3-30 minutes.

Centrifuge the block for 10-15 minutes at about 3200 RCF at RT.

Remove an aliquot of supernatant and dilute at about 1:10 in the dilution buffer. For example, transfer 15 μl of supernatant of the supernatant to a new 96 well plate containing 135 μl of the dilution buffer and mix.

The DNA is ready for qPCR, with or without further dilution.

The DNA is ready for PCR and can be saved at 4° C. or −20° C.

Alternatively, incubation of the DNA extraction solute for 3 to 30 minutes may be excluded from the process, whereby incubating the solute would consist of the 1 minute shaking in the grinder. Alternatively, the term “incubation” or “incubating” includes within its scope shaking the solute for one minute followed by incubating the solute for a period of time, e.g. 3 to 30 minutes. Alternatively, incubating can mean eliminating the shaking step and incubating the DNA extraction solute for a period of time. It is understood, that incubating the solute can be done in any number of ways, including different incubation times and at different temperatures for the purpose of extracting gDNA from the tissue or cell sample and are all referred to herein as the DNA extraction step. All these various alternatives are considered encompassed by the terms “incubated,” “incubating,” and “DNA extraction step.”

In the dilution step of the invention, the pH of the supernatant containing the DNA is neutralized by diluting with a buffer, e.g., Tris-HCl, whereby the supernatant is neutralized to preferably within a range of approximately 8.0-9.0 which pH is compatible with a PCR buffering system.

When adding a buffer to the alkali dominated supernatant, the pH of the supernatant will be reduced to a lower value depending on relative amounts of them. By way of example, 1 volume of supernatant is mixed with 9 volumes (or 4 volumes) of the dilution/neutralization buffer so the pH of the mixtures will be lowered to 8.0-9.0, which corresponds to the optimal pH for subsequent PCR analysis.

The dilution buffer comprises about 5 mM-200 mM Tris-HCl (about pH5-pH9).

The dilution buffer comprises preferably 40 mM Tris-HCl, pH7.5.

The dilution buffer can also include 0.1 mM to 5 mM EDTA

The dilution buffer comprises about 0%-1% PVP-40.

In a preferred embodiment, the dilution buffer comprises 5 mM to 200 mM Tris-HCl, 0.1 mM to 5 mM EDTA, and optionally 1% or less of PVP-40 in water.

In a more preferred embodiment, the dilution buffer comprises 40 mM Tris-HCl (pH7.5), 1 mM EDTA.

An even more preferred embodiment, the dilution buffer comprises 40 mM Tris-HCl (pH7.5), 1 mM EDTA, and 0.5% PVP-40.

The method of the invention comprises extracting genomic DNA from disrupted plant or animal tissue or cells comprising; adding to the tissue or cells an DNA extraction reagent comprising an alkaline component, a detergent, a salt and optionally a polyphenol absorbing compound to form a DNA extraction solute, incubating the tissue DNA extraction solute in a DNA extraction step, wherein the DNA extraction solute is kept at pH of at least 10, of at least 11, of at least 12, of at least 13, or at about 14 substantially throughout the extraction step; and using the supernatant containing gDNA in a DNA amplification process.

The method of extracting genomic DNA from plant or animal tissue or cells further includes the step of diluting the DNA extraction solute in a neutralizing buffer, wherein the supernatant of the DNA extraction solute is diluted at least 5 fold, preferably 10 fold.

The method of the invention comprises extracting genomic DNA from plant or animal tissue or cells comprising; adding to the disrupted tissue and/or disrupted cells a DNA extraction reagent comprising an alkaline component, a detergent, a salt and optionally a polyphenol absorbing compound to form a DNA extraction solute; incubating the tissue DNA extraction solute in a DNA extraction step, wherein the DNA extraction solute is kept at pH of at least 10, at least 11, at least 12, at least 13, or at about 14 substantially throughout the extraction step; centrifuging the reagent to clarify the supernatant and using the supernatant containing the extracted gDNA for analysis or in a DNA amplification process.

Methods for Analyzing and/or Employing Isolated DNA

The presently disclosed subject matter also provides methods for analyzing gDNA prepared using the methods disclosed herein, as well as for employing the gDNA so prepared in one or more downstream applications. Any analytical and/or other downstream technique that can be employed on gDNA could be performed with the gDNA isolated by the presently disclosed methods using the compositions disclosed herein. The following includes a non-limiting listing of exemplary analytical and/or other downstream techniques for which the gDNA isolated by the presently disclosed methods using the compositions disclosed herein would be appropriate.

Methods for PCR, Including Quantitative PCR (qPCR)

In some embodiments, the presently disclosed subject matter provides methods for performing PCR, including but not limited to qPCR, using gDNA prepared by the presently disclosed methods. In some embodiments, the methods comprise providing a gDNA sample prepared by the presently disclosed method and performing PCR under conditions wherein the Ct value can be collected in real-time for quantitative analysis, whereby qPCR of the gDNA sample is performed.

As used herein, the phrase “Ct value” refers to “threshold cycle”, which is defined as the “fractional cycle number at which the amount of amplified target reaches a fixed threshold”. In some embodiments, it represents an intersection between an amplification curve and a threshold line. The amplification curve is typically in an “S” shape indicating the change of relative fluorescence of each reaction (Y-axis) at a given cycle (X-axis), which in some embodiments is recorded during PCR by a real-time PCR instrument. The threshold line is in some embodiments the level of detection at which a reaction reaches a fluorescence intensity above background. See Livak & Schmittgen (2001) 25 Methods 402-408. It is a relative measure of the concentration of the target in the PCR. Generally, good Ct values for quantitative assays such as qPCR are in some embodiments in the range of 10-40 for a given reference gene.

Additionally, good Ct values for quantitative assays such as qPCR show a linear response range with proportional dilutions of target gDNA.

In some embodiments, qPCR is performed under conditions wherein the Ct value can be collected in real-time for quantitative analysis. For example, in a typical quantitative PCR experiment, DNA amplification is monitored at each cycle of PCR during the extension stage. The amount of fluorescence generally increases above the background when DNA is in the log linear phase of amplification. In some embodiments, the Ct value is collected at this time point.

Methods for Performing Genome Analysis

The presently disclosed subject matter also provides in some embodiments methods of performing genome analysis, including but not limited to genetic marker analysis, such as but not limited to genetic marker analysis related to molecular marker assisted breeding and/or selection; locus copy number analysis; zygosity analysis; seed purity assessment based on molecular marker profiles; and/or plant pathogen and/or disease control. In some embodiments, the methods comprise providing a gDNA sample prepared by the presently disclosed method and performing detecting the presence of a genetic marker (including, but not limited to a single nucleotide polymorphism; SNP) present in the gDNA sample. In some embodiments, the detecting methodology includes a PCR reaction on the gDNA sample prepared by the presently disclosed method, wherein the PCR reaction employs one or more oligonucleotide primers designed to detect the presence or absence of a genetic marker of interest.

In some embodiments, the PCR reaction is qPCR employed for determining copy number of a genetic marker in the genome of an individual. Generalized techniques for assessment of copy number can be found in Livak & Schmittgen (2001) 25 Methods 402-408, Abad et al. (2010) 5 Biotechnol J 412-420, D'haene et al. (2010) 50 Methods 262-270, Ji et al. (2012) 14 J Mol Diagnostics 280-285, etc. T GDNA prepared using the compositions and methods of the presently disclosed subject matter can be employed in these exemplary methods. In some embodiments, the assessment of copy number is in the context of determining the number of copies of a transgene in a transgenic cell and/or subject, and in some embodiments the assessment of copy number is in the context of determining the amplification or the loss of a genetic locus such as, but not limited to a gene.

In some embodiments, gDNA prepared using the compositions and methods of the presently disclosed subject matter can be employed in zygosity analysis. As used herein, the phrase “zygosity analysis” refers to any technique that can be used to determine whether a cell, tissue, organ, or a subject is nullizygous, hemizygous, heterozygous, or homozygous for a particular nucleic acid sequence of interest. Generalized techniques for zygosity analysis include, but are not limited to those disclosed in Tesson et al. (2010) 597 Methods Mol Biol 277-285. For example, the gDNA prepared using the compositions and methods of the presently disclosed can be used in subsequent PCR with target- and/or allele-specific assays in zygosity analysis, wherein the DNA prepared using the presently disclosed compositions and/or methods can serve as template for PCR amplification.

In some embodiments, gDNA prepared using the compositions and methods of the presently disclosed subject matter can be employed in analyzing seed purity based on molecular marker profiles. By way of example and not limitation, the gDNA prepared using the compositions and methods of the presently disclosed subject matter can be employed in a molecular marker analysis, and the purity of a given collection of seeds can be determined by identifying whether or not the seeds constitute a single molecular marker profile or if multiple profiles can be identified. In the latter instance, the presence of multiple marker profiles or SNP genotype profiles for one or more selected genes can suggest either heterozygosity in the seeds (e.g., two different alleles at a given locus are present in the seeds in equal proportions), whereas the presence of multiple marker profiles for one or more selected genes can also suggest that the seeds are not isogenic (i.e., comprise a plurality of genomes).

In some embodiments, gDNA prepared using the compositions and methods of the presently disclosed subject matter can be employed in or plant pathogen and/or disease detection and/or monitoring.

### Example 1

### Optimizing Reagent Concentrations

A series of experiments were performed in which various concentrations of NaOH and ammonium acetate (NH₄Ac) comprising the DNA extraction reagent and were employed in the preparation of gDNA from leaf tissues of maize. The following describes the general procedure that was used for each test.

Securely seal sample block containing 1 steel bead and 4 leaf discs/well frozen or lyophilized. Each disc is approximately 6 mm in diameter.

Shake the block for 2 minutes in a paint shaker or equivalent grinder, such as a Kleco grinder.

Centrifuge the block for 1 minute at about 3200 RCF at RT.

Dispense 200 μl of DNA extraction reagent into each well to form the DNA extraction solute. Re-seal the block and shake for about 1 minute in the grinder.

Incubate the solute at RT for 3-30 minutes.

Centrifuge the block for 10-15 minutes at about 3200 RCF at RT.

Remove an aliquot of supernatant and dilute at about 1:10 in the dilution buffer. For example, transfer 15 μl of supernatant of the supernatant to a new 96 well plate containing 135 μl of the dilution buffer and mix.

The DNA is ready for qPCR, with or without further dilution.

The DNA is ready for PCR and can be saved at 4° C. or −20° C.

The DNA that can be extracted from 4 corn leaf discs using the DNA extraction reagent and method of the invention is in the amount of at least 4,000 ng, wherein the total DNA that can be extracted from 4 soybean leaf discs is at least 5,000 ng, determined by qPCR analysis. It is within the scope of the invention to recover up to 10,000 ng of DNA from numerous different plant tissues and plant species from similar amount of tissue sample.

### Example 2

The impact of different concentrations of NaOH and dilution on Ct value in qPCR

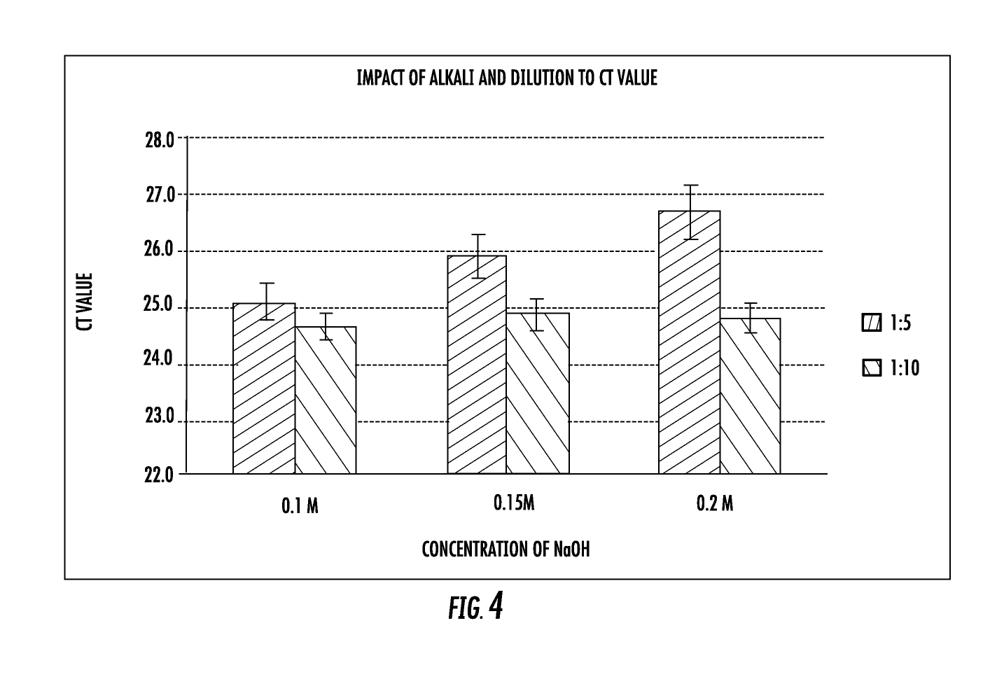

FIG. 4 shows the impact of different concentrations of NaOH and dilution of the DNA extraction reagent of the invention on the Ct value for qPCR. Each condition is replicated in qPCR to obtain reliable Ct values for extraction of genomic DNA from plant tissue. NaOH in the DNA extraction reagent was at 0.1M, 0.15, and 0.2, each diluted 1:5 and 1:10, wherein the other components of the DNA extraction reagent were fixed at 0.1% SDS, 0.3M NH₄Ac, and 1% PVP-40. At these ranges the extraction reagent and method of the invention delivered high quantity of high quality DNA for qPCR. The experiments do show that NaOH at a 1:10 dilution is better than a 1:5 dilution especially at relatively high NaOH concentration. It was found that there is no significant difference between the two dilutions at 0.1M NaOH, wherein the Ct values obtained were under 26. As low as a 1:5 dilution of the supernatant results in DNA extracts suitable for qPCR analysis, showing the DNA extraction reagent and method of the invention is robust under a range of conditions for producing high quality and high quantity DNA for PCR amplification for qPCR and other DNA amplification methods requiring high quality and high quantity DNA.

### Example 3

The impact of changes to NH₄Ac concentration and dilutions in qPCR

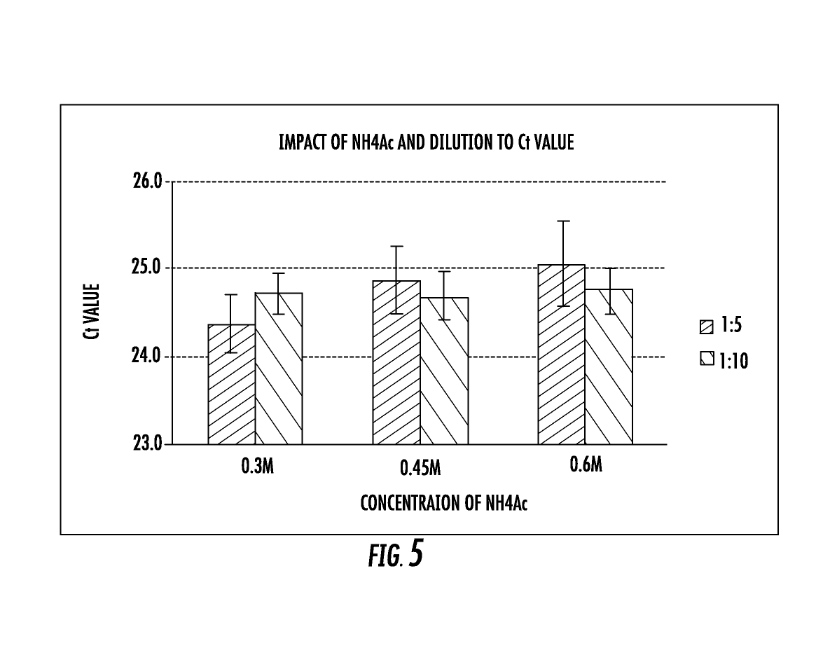

FIG. 5 shows data on the impact of changes to NH₄Ac concentration of the DNA extraction reagent and dilutions thereof on the Ct value, wherein the other DNA extract reagent components are fixed at 0.1M NaOH, 0.1% SDS and 1% PVP-40. In these set of experiments, NH₄Ac was used at concentrations of 0.3M, 0.45M, and 0.6M, each at a dilution of 1:5 and 1:10. At these ranges, all Ct values are low and consistent, showing the DNA extraction reagent and method of the invention is robust under these ranges of conditions for producing high quality and high quantity DNA for qPCR and other DNA amplification methods requiring high quality and high quantity DNA.

### Example 4

### Impact of Supernatant Dilutions on Ct Value in qPCR

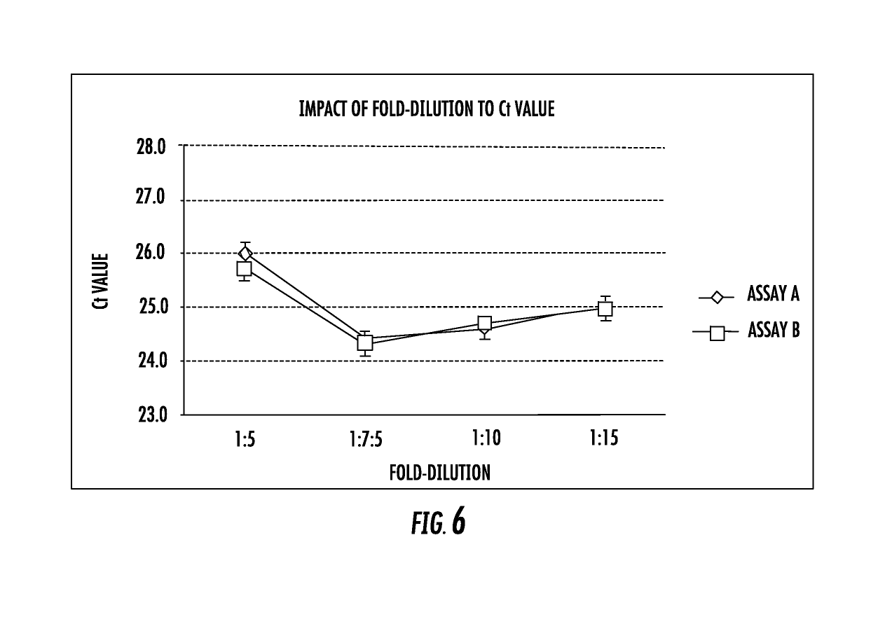

FIG. 6 shows the impact various supernatant dilutions on the Ct value. DNA extraction reagent used to extract the genomic DNA was 0.1M NaOH, 0.1% SDS, 0.3M NH₄Ac, and 1% PVP-40. The data shows that the Ct value of the supernatant dilutions were in a linear dose-response range from as low as dilution 1:7.5, which represents a DNA extract of high quality. More importantly, the Ct values fall into a linear dose-response range at and after dilutions 1:7.5 (e.g. 1:10, 1:20, 1:40, and 1:80).

### Example 5

In this Example, the DNA extraction reagent of the invention comprises 0.1M NaOH, 0.1% SDS, 0.3M NH₄Ac, and 1% PVP-40. (e.g., 1000 ml =20 ml 5M NaOH, 5 ml 20% SDS, 40 ml 7.5M NH₄Ac, and 10 g PVP-40 and add distilled water to 1000 ml).

The dilution buffer of the dilution buffer comprises 40 mM Tris-HCl, pH7.5; 1 mM EDTA, and 0.5% PVP-40. (e.g., 1000 ml =20 ml 2M Tris-HCl, pH7.5, 2 ml 0.5M EDTA, pH8.0, 5 g PVP-40 and add distilled water to 1000 ml).

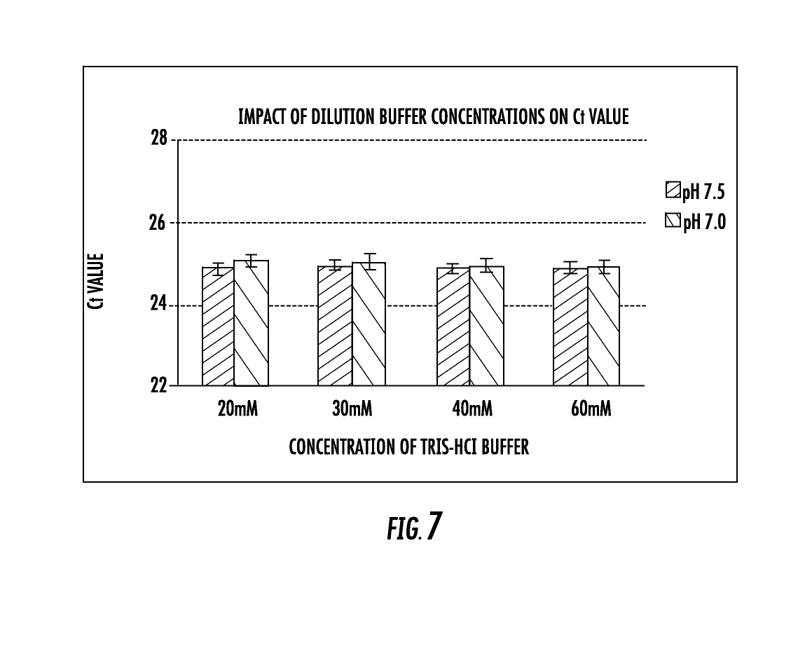

FIG. 7 shows the impact of on Ct value of different Tris-HCl dilution buffer concentrations for a 1:10 dilution of the supernatant. 40 mML Tris-HCl at pH7.5 was selected as the preferred condition. However, all depicted concentrations of Tris-HCl dilution buffer worked well at each pH.

Using the preferred DNA extraction reagent comprising 0.1M NaOH, 0.1%, SDS, 0.3M NH₄Ac, and 1% PVP-40, DNA was extracted from corn tissue and the supernatant was diluted 1:10 in 40 mM Tris-HCl (pH7.5). 9586 data points were generated and analyzed. Only twenty six data points were missing. This translates into a very low percentage (0.27%) of missing data, thereby confirming that the invention generates high quality, high quantity gDNA for qPCR or other DNA amplification methods requiring high quality and high quantity of DNA.

Further embodiments of the invention include extracting gDNA from sugarcane, sunflower, pepper, and tomato. All experiments on these crops obtained extracted gDNA of a quantity and quality equivalent to the gDNA extracted from corn and soybean as described in the above. Examples and FIGS. 4-7. Real-time quantitative results were obtained give Ct values in the range of 20 to 26 at 1:10 dilution of the supernatant of the DNA extraction solute, indicative of the high quality of the DNA extracted and demonstrating the broad applicability of the presently disclosed methods to a number of plant and tissue types.

Additionally, the presently disclosed methods provide further advantages relative to standard gDNA preparation methods. First, the presently disclosed methods can be employed for most if not all sources of biological materials, and yield gDNA that is of high quality and has been shown to be stable for at least 8 weeks when stored at 4° C. The presently disclosed methods are also easily adaptable to high-throughput procedures and are very economical, using reagents that are relatively inexpensive. And finally, the reagents employed are user and environment friendly.

### Protocol for Extracting DNA from Cell Cultures

Pellet cultured cells of about 1,000-5,000,000 per well in 96-well plate by centrifugation for 3 minutes at 1500 RPM at RT.

Discard culture medium and add 100 μl of DNA extraction reagent to each well.

Seal the plate and vortex at high speed for 1 minute to form the DNA extraction solute.

Incubate the solute at RT for 3-30 minutes.

Centrifuge the plate for 10 minutes at 4000 RPM at RT.

Dilute the supernatant containing DNA at 1:10 in the dilution buffer in a new plate.

The DNA is ready for qPCR analysis, with or without further dilution, or for storage in 4° C. after the plate is sealed.

While the present invention has been described in terms of exemplary embodiments, the skilled artisan will readily understand that numerous variations and modifications of these exemplary embodiments are possible without undue experimentation. All such variations and modifications are within the intended scope of the claimed invention.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Dellaporta et al. (1983) A plant DNA minipreparation: Version II Plant Molecular Biology Reporter, Vol 1, No. 4
Sambrook & Russell, 2001 Molecular Cloning, A Laboratory Manual (3rd Edition). Cold Spring Harbor Laboratory Press, Cold Spring Harbor, N.Y.
Ausubel et al. (eds.) (1988) Current Protocols in Molecular Biology. Greene Pub. Associates; Wiley-Interscience, New York, N.Y., United States of America
Ausubel et al. (eds.) (1999) Short Protocols in Molecular Biology (Fourth Edition). John Wiley & Sons, New York, N.Y., United States of America
Sambrook & Russell (2001) Molecular Cloning, A Laboratory Manual (Third Edition). Cold Spring Harbor Laboratory Press, Cold Spring Harbor, N.Y., United States of America
Murray & Thompson (1980) Rapid isolation of high molecular weight plant DNA. Nucleic Acids Res 8:4321-4326 Kotchoni & Gachomo (2009) Mol Biol Rep 36:1633-1636
Dilworth & Frey (2000) A rapid method for high throughput DNA extraction from plant material for PCR amplification. Plant Mol Biol Rep 18:61-64
Wang et al. (1993) A simple method of preparing plant samples for PCR, Nucleic Acids Research, 21, 4153-4154
Porcar et al. (2007) A simple DNA extraction method suitable for PCR detection of genetically modified maize. J Sci Food Agric 87:2728-2731
Post et al. (2003) A highu-throughput DNA extraction method for barley seed, Euphytica 130:255-260
Paris, Carter (2000) Cereal DNA: A rapid High-Throughput Extraction Method for Marker Assisted Selection, Plant Mol. Biol Reporter 18:357-360
Montero-Pau (2008) Application of an inexpensive and high-throughput genomic DNA extraction method for the molecular ecology of zooplanktonic diapausing eggs, Limnol. Oceanogr, Methods 6:218-222
Sharma et al. (2013), An improved method of DNA extraction from plants for pathogen detection and genotyping by polymerase chain reaction, African Journal of Biotechnology, V12(15), pp 1894-1901
Osmundson et al. (2013) Back to basics: an evaluation of NaOH and alternative rapid DNA extraction protocols for DNA barcoding, genotyping, and disease diagnostics from fungal and oomycete samples, Molecular Ecology Resources 13:66-74
Wen & Deng (2002) The extraction of gDNA from five species of Rosa. Seed 126:18-21 Livak & Schmittgen (2001) 25 Methods 402-408
Qiang et al. (2004) A simple protocol for isolating gDNA from Chestnut Rose (Rosa roxburghii Tratt) for RFLP and PCR Analysis. Plant Mol Biol Rep 22:301-302
Michiels et al. (2003) Extraction of high quality gDNA from latex containing plants. Anal Biochem 315:85-89
WO2014/018195 Lysis buffer and methods for extraction of DNA from plant material
Abad et al. (2010) 5 Biotechnol J 412-420
D'haene et al. (2010) 50 Methods 262-270
Ji et al. (2012) 14 J Mol Diagnostics 280-285

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A DNA extraction reagent comprising an alkali, a surfactant, and a salt, wherein the pH of the reagent is at least 10.

2. The DNA extraction reagent of claim 1, wherein the alkali is NaOH.

3. The DNA extraction reagent of claim 1, wherein the surfactant is SDS.

4. The DNA extraction reagent of claim 1, wherein the salt is NH₄Ac.

5. The DNA extraction reagent of claim 1, further comprising a polyphenol absorbing compound.

6. The DNA extraction reagent of claim 5, wherein the polyphenol absorbing compound is PVP-40

7. The DNA extraction reagent according to claim 1, wherein the alkali is NaOH at a concentration of at least 0.1M, the surfactant is SDS at a concentration of at least 0.1%, and the salt is NH₄Ac at a concentration of at least 0.3M.

8. The DNA extraction reagent according to claim 1, wherein the pH of the reagent is at least 11.

9. The DNA extraction reagent according to claim 1, wherein the pH of the reagent is at least 12

10. The DNA extraction reagent according to claim 7, wherein the pH of the reagent is at least 10.

11. The DNA extraction reagent according to claim 7, wherein the pH of the reagent is at least 11.

12. The DNA extraction reagent according to claim 7, wherein the pH of the reagent is at least 12

13. A DNA extraction reagent comprising:
a. an alkali selected from the group consisting of potassium hydroxide, (KOH), metal hydroxide, alkali salts, and sodium hydroxide (NaOH), and combinations thereof;
b. a surfactant selected from the group consisting of SDS, Triton X-100, Triton X-114, NP-40, Tween 20, Tween 80, Octyl glucoside, Octyl thioglucoside, CHAPS, and combinations thereof;
c. a salt selected from the group consisting of sodium chloride (NaCl), potassium chloride (KCl), potassium phosphates, sodium bicarbonate (NaHCO₃), sodium acetate (C₂H₃NaO₂), and ammonium acetate (NH₄C₂H₃O₂), and combinations thereof; and
wherein the pH of the DNA extraction reagent is at least 10.

14. The DNA extraction reagent according to claim 13, wherein the alkali in the DNA extraction reagent is at least 0.1M.

15. The DNA extraction reagent according to claim 13, wherein the concentration of surfactant in the DNA extraction reagent is at least 0.1%.

16. The DNA extraction reagent according to claim 13, wherein the concentration of the salt in the DNA extraction reagent is at least 0.3M.

17. The DNA extraction reagent according to claim 13, wherein the pH is at least 11.

18. The DNA extraction reagent according to claim 13, wherein the pH is at least 12.

19. A method for extracting genomic DNA from plant or animal tissue or cells comprising;
a. disrupting the tissue or cells;
b. adding to the disrupted tissue or cells a DNA extraction reagent comprising an alkali, a detergent, a salt and optionally a polyphenol absorbing compound to form a DNA extraction solute;
c. incubating the DNA extraction solute in a DNA extraction step, wherein the DNA extraction solute is kept at pH of at least 10;
d. spinning the DNA extraction solute to form a supernatant; and
e. using the supernatant containing DNA in a DNA amplification process.

20. A method for extracting genomic DNA of claim 19, wherein the alkali is selected from the group consisting of potassium hydroxide, (KOH), metal hydroxide, alkali salts, and sodium hydroxide (NaOH), and combinations thereof.

21. A method for extracting genomic DNA of claim 19, wherein the surfactant is selected from the group consisting of SDS, Triton X-100, Triton X-114, NP-40, Tween 20, Tween 80, Octyl glucoside, Octyl thioglucoside, CHAPS, and combinations thereof.

22. A method for extracting genomic DNA of claim 19, wherein the salt is selected from the group consisting of sodium chloride (NaCl), potassium chloride (KCl), potassium phosphates, sodium bicarbonate (NaHCO₃), sodium acetate (C₂H₃NaO₂), and ammonium acetate (NH₄C₂H₃O₂), and combinations thereof.

23. A method for extracting the genomic DNA of claim 19, wherein disrupting tissues or cells is done without the addition of liquid media.

24. A method for extracting the genomic DNA of claim 19, wherein the supernatant is diluted in a neutralizing buffer.

25. A method for extracting the genomic DNA of claim 19, wherein the supernatant is diluted 1:5 or less in a neutralizing buffer.

26. A method for extracting the genomic DNA of claim 19, wherein the supernatant is diluted 1:10 or less in a neutralizing buffer.

27. A method for extracting the genomic DNA of claim 19, wherein the supernatant is diluted less than 1:100, less than 1:75, less than 1:50, less than 1:30, or less than 1:20 in a neutralizing buffer.

28. A method for extracting genomic DNA from animal or plant tissue or cells comprising;
a. adding to the tissue or cells a DNA extraction reagent comprising an alkali, a detergent, and a salt to form a DNA extraction solute;
b. incubating the DNA extraction solute in a DNA extraction step, wherein the tissue DNA extraction solute is kept at pH of at least 10;
c. spinning the DNA extraction solute to form a supernatant containing the gDNA; and
d. diluting the supernatant in a neutralizing buffer.

29. A method for extracting the genomic DNA of claim 28, further comprising using the supernatant diluted in the neutralizing buffer in qPCR.

30. A method for extracting the genomic DNA of claim 28, wherein disrupting tissues or cells is done without the addition of liquid media.

31. A method for extracting the genomic DNA of claim 28, wherein the supernatant is diluted 1:5 or less in a neutralizing buffer.

32. A method for extracting the genomic DNA of claim 28, wherein the supernatant is diluted about 1:10 or less in a neutralizing buffer.

33. A method for extracting the genomic DNA of claim 28, wherein the supernatant is diluted about 1:5 to 1:10 in a neutralizing buffer.

34. A method for extracting the genomic DNA of claim 28, wherein the supernatant is diluted less than 1:100, less than 1:75, less than 1:50, less than 1:30, or less than 1:20 in a neutralizing buffer.
